(19) United States
(12) Patent Application Publication
(10) Pub. No.: US 2015/0329505 A1
(43) Pub. Date: Nov. 19, 2015

(54) A FLUORESCENT COMPOSITION FOR USE IN MARKING OBJECTS, SUCH AS FLOWERS

(71) Applicant: BIOCONST PTY LTD, Glenelg, South Australia (AU)

(72) Inventors: Mark Alfred Tester, Linden Park (AU); Harshavardani PADMANABHAN, Colonel Light Gardens (AU)

(21) Appl. No.: 14/375,744

(22) PCT Filed: Jan. 31, 2013
(86) PCT No.: PCT/AU2013/000079

§ 371 Date: Jul. 30, 2014

(30) Foreign Application Priority Data
Jan. 31, 2012 (AU) 2012900346

(51) Int. Cl. C07D 263/64; C07C 309/38; C09K 11/06

(52) U.S. Cl. CPC C07D 263/64; C09K 11/06; C07C 309/38; C09K 2211/1088; C09K 2211/1011

(57) ABSTRACT

The present invention relates to a fluorescent composition, comprising polycyclic aromatic fluorophores, and its use in marking objects, such as plants. The composition may be applied to the surface of the plant or it can be taken up by the plant providing luminescent flower.

### PRIORITY CLAIM

This application claims priority to Australian provisional patent application 2012900346 filed on 31 Jan. 2012, the content of which is hereby incorporated by reference.

### FIELD

The present disclosure relates to a fluorescent composition, use of a fluorescent composition, and to objects, kits and combinations products comprising a fluorescent composition.

### BACKGROUND

There are many situations where it may be desirable to mark a product with a fluorescent agent. For example, the decorative appeal of some products is improved if all or part of the product can fluoresce under appropriate light conditions. Examples of such products include novelty items and ornamental organisms, such as plants and flowers.

In other circumstances, it may be desirable to mark a product with a fluorescent agent, for example so as to tag or identify the product. For example, products may be marked with a fluorescent agent so that the product fluoresces with a selected wavelength upon irradiation with light of a wavelength able to excite the fluorophore, thereby allowing identification of the product.

In circumstances where a fluorescent agent is added to a product so as to mark the product, the selection of the fluorescent properties of the fluorescent agent is an important consideration. For example, the excitation and emission characteristics of the fluorescent agent need to be matched with the properties of the product and its desired use.

The selection of the vehicle used in conjunction with the fluorescent agent may also be an important consideration. The selection of the vehicle is particularly important in relation to the required means of application/introduction and/or the properties of the fluorescent agent. For example, if the agent is to be applied by way of spray application, the properties of the liquid vehicle are important in relation to droplet size produced upon spraying.

In circumstances where a product is marked by application of a fluorescent agent to a surface of product, another important consideration is the properties of the surface of the product to which the fluorescent agent is to be applied. For example, some surfaces are inherently difficult to mark. This is particularly the case for surfaces that are predominantly hydrophobic or hydrophilic. The wettability of surfaces also varies greatly.

Accordingly, there is a need to provide marking compositions that address one or more problems in the art and/or provide one or more advantages in the art.

### SUMMARY

The present disclosure relates to a fluorescent composition and uses thereof.

Certain embodiments of the present disclosure provide a fluorescent composition comprising a polycyclic aromatic fluorophore.

Certain embodiments of the present disclosure provide use of a fluorescent composition comprising a polycyclic aromatic fluorophore for marking an object.

Certain embodiments of the present disclosure provide use of a fluorescent composition comprising a polycyclic aromatic fluorophore for uptake by a plant and/or flower.

Certain embodiments of the present disclosure provide an object comprising a fluorescent composition comprising a polycyclic aromatic fluorophore.

Certain embodiments of the present disclosure provide an arrangement of one or more flowers and/or plants, the one or more flowers and/or plants comprising a fluorescent composition comprising a polycyclic aromatic fluorophore, the fluorescent composition applied to a surface of the one or more flowers and/or plants.

Certain embodiments of the present disclosure provide an arrangement of one or more flowers and/or plants, the one or more flowers and/or plants comprising a fluorescent composition comprising a polycyclic aromatic fluorophore, the fluorescent composition being taken up by the one or more flowers and/or plants.

Certain embodiments of the present disclosure provide a kit comprising a fluorescent composition comprising a polycyclic aromatic fluorophore.

Certain embodiments of the present disclosure provide a combination product, the combination product comprising the following components:

a fluorescent composition comprising a polycyclic aromatic fluorophore; and

an atomizing device.

Certain embodiments of the present disclosure provide a method of producing a fluorescent plant and/or or a part thereof, the method comprising introducing a fluorescent composition comprising a polycyclic aromatic fluorophore into the plant and/or a part thereof by uptake.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows flowers sprayed with 0.1% (w/v) pyranine solution at a pH of 3.0, when excited by UV light.

FIG. 2 shows flowers sprayed with 0.1% (w/v) pyranine solution at a pH of 6.84, when excited by UV light.

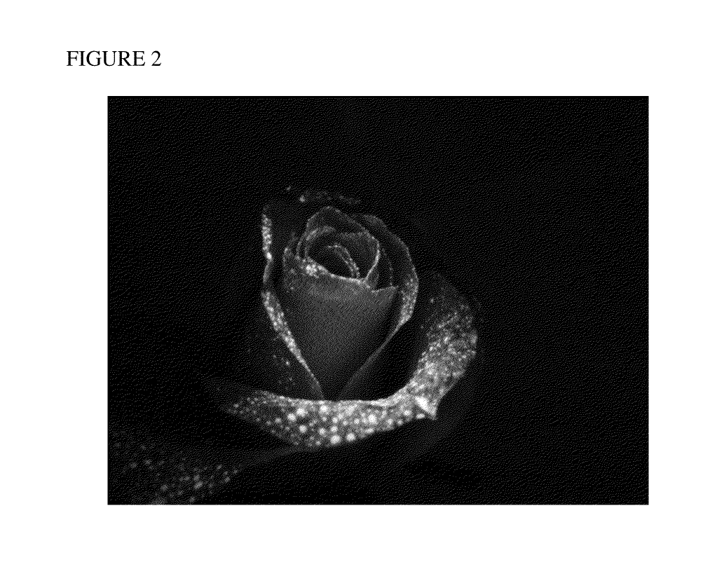

FIG. 3 shows a rose flower after uptake of a solution of 0.1% pyranine, when excited by UV light.

FIG. 4 shows a chrysanthemum flower after uptake of a solution of 0.1% pyranine, when excited by UV light.

FIG. 5 shows a chrysanthemum flower after uptake of a solution of 0.1% pyranine, when excited by UV light.

FIG. 6 shows use of a red formulation comprising 0.07% Eu, 2-thenoyltrifluoroacetone triphenylphosphine oxide on a rose flower when excited by UV light.

FIG. 7 shows use of a red formulation comprising 0.07% Eu, 2-thenoyltrifluoroacetone triphenylphosphine oxide on a chrysanthemum flower when excited by UV light.

FIG. 8 shows use of a blue formulation comprising 0.03% 4,4′-bis(2-benzoxazolyl)stilbene on a chrysanthemum flower when excited by UV light.

### DETAILED DESCRIPTION

The present disclosure relates to a fluorescent composition and uses thereof.

Certain embodiments of the present disclosure are directed to a fluorescent composition, use of a fluorescent composition, objects comprising a fluorescent composition, and kits and combination products comprising a fluorescent composition.

Certain disclosed embodiments provide fluorescent compositions, use of fluorescent compositions, objects comprising a fluorescent composition, and kits and combination products comprising a fluorescent composition that have one or more advantages. For example, some of the advantages of certain embodiments disclosed herein include one or more of the following: a composition compatible with marking a range of different types of products; a composition that provides aesthetic appeal when fluorescence occurs; a composition that does not promote wilting and/or reduce vase life of plants when applied; a composition that produces droplets of a small size when the composition is sprayed or atomized; a composition that is able to produce fine droplets on waxy and/or non-waxy surfaces that have a reduced tendency to spread over the surface; and a composition that may be taken up by a plant to allow some parts of the plant to fluoresce. Other advantages of certain embodiments of the present disclosure are disclosed herein.

Certain embodiments of the present disclosure provide a fluorescent composition comprising a polycyclic aromatic fluorophore.

The present disclosure is based, at least in part, on the recognition that a composition comprising a fluorophore based on polycyclic aromatic hydrocarbons provides one or more advantages: (i) the fluorescent properties of fluorophores based on polycyclic aromatic hydrocarbons are compatible with marking a range of different types of products; (ii) the emission wavelengths of fluorophores based on polycyclic aromatic hydrocarbons provide aesthetic appeal; (iii) the excitation wavelengths are of a suitable range for many commercially available UV-emitting devices; and (iv) fluorophores based on polycyclic aromatic hydrocarbons when applied or introduced to products such as flowers do not promote wilting and/or reduce vase life, unlike other types of fluorescent agents. Other advantages are disclosed herein.

The term “fluorophore” refers to any agent, molecule, compound, ion, or element that directly and/or indirectly has the capacity to fluoresce and/or cause fluorescence.

The term “polycyclic aromatic fluorophore” refers to a fluorophore that comprises two or more aromatic cyclic groups. For example, the fluorophore may comprise (i) cyclic aromatic groups that share common atoms (eg a naphthalene or a pyrene based fluorophore); separate cyclic aromatic groups that are chemically bonded together directly or indirectly (eg a stilbene based fluorophore); and (iii) cyclic aromatic groups that are directly or indirectly complexed to a metal ion (eg Europium or other rare earth complexed fluorophores). Typically, the properties of the cyclic aromatic groups will contribute to the fluorescence of the fluorophore, however it should understood that the polycyclic aromatic fluorophores of the present disclosure are not so limited.

Examples of aromatic cyclic groups include one or more of the following: furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, benzene, naphthalene, anthracene, benzopyrone, pyridine, quinoline, isoquinoline, pyrazine, quinoxaline, acridine, pyrimidine, quinazoline, pyridazine, cinnoline, phthalazine, 1,2,3-triazine, 1,2,4-triazine, 1,3,5-triazine, and derivatives (including substituted derivatives) of any of the aforementioned. Other aromatic cyclic groups are contemplated.

In certain embodiments, the polycyclic aromatic fluorophore comprises one or more cyclic aromatic groups selected from one or more of furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, benzene, naphthalene, anthracene, benzopyrone, pyridine, quinoline, isoquinoline, pyrazine, quinoxaline, acridine, pyrimidine, quinazoline, pyridazine, cinnoline, phthalazine, 1,2,3-triazine, 1,2,4-triazine, 1,3,5-triazine, and/or a substituted derivative of any of the aforementioned.

In certain embodiments, the fluorescent composition comprises one or more fluorophores. In certain embodiments, the fluorescent composition comprises a single fluorophore. In certain embodiments, the fluorescent composition comprises a plurality of fluorophores. In certain embodiments, the fluorescent composition comprises at least one fluorophore. In certain embodiments, the fluorescent composition comprises at least two fluorophores. In certain embodiments, the fluorescent composition comprises a combination of fluorophores.

In certain embodiments, the fluorophore comprises an excitation wavelength of 750 nm or less, 620 nm or less, 590 nm or less, 570 nm or less, 500 nm or less, 495 nm or less, 490 nm or less, 480 nm or less, 475 nm or less, 470 nm or less, 460 nm or less, 450 nm or less, 440 nm or less, 430 nm or less, 420 nm or less, 410 nm or less, 400 nm or less, 390 nm or less, 380 nm or less, or 370 nm or less. In certain embodiments, the fluorophore comprises an excitation wavelength in the ultraviolet range or near ultraviolet range. In certain embodiments, the fluorophore comprises an excitation wavelength of 400 nm or less. In certain embodiments, the fluorophore comprises an excitation wavelength of about one of the aforementioned wavelengths.

The term “about” or means an acceptable error for a particular value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurements method. For removal of doubt, it shall be understood that any range stated herein that does not specifically recite the term “about” before the range or before any value within the stated range inherently includes such term to encompass the approximation within the deviation noted above.

In certain embodiments, the fluorophore comprises an excitation wavelength in the range of red light (620-750 nm), orange light (590-620 nm), yellow light (570-590 nm), green light (495-570 nm), cyan light (476-495 nm), blue light (450-475 nm), violet light (380-450 nm), ultraviolet A (UVA; 315-400), near ultraviolet light (300-400 nm), or 360 nm-470 nm. In certain embodiments, the fluorophore comprises an excitation wavelength in the range of 350-400 nm. In certain embodiments, the fluorophore comprises an excitation wavelength of about one of the aforementioned ranges.

The term “excitation wavelength” refers to light having a wavelength that falls within the excitation spectrum of a fluorophore.

In certain embodiments, the fluorophore comprises an emission wavelength (upon irradiation with light of an excitation wavelength) of 750 nm or less, 620 nm or less, 590 nm or less, 570 nm or less, 500 nm or less, 495 nm or less, 490 nm or less, 480 nm or less, 475 nm or less, 470 nm or less, 460 nm or less, 450 nm or less, 440 nm or less, 430 nm or less, 420 nm or less, 410 nm or less, 400 nm or less, 390 nm or less, 380 nm or less, or 370 nm or less. In certain embodiments, the fluorophore comprises an emission wavelength of about one of the aforementioned wavelengths.

In certain embodiments, the fluorophore comprises an emission wavelength in the range of red light (620-750 nm), orange light (590-620 nm), yellow light (570-590 nm), green light (495-570 nm), cyan light (476-495 nm), blue light (450-475 nm), or violet light (380-450 nm). In certain embodiments, the fluorophore comprises an emission wavelength of about one of the aforementioned wavelengths.

In certain embodiments, the fluorophore comprises an emission wavelength in the range from 490 to 560 nm upon irradiation with light of an excitation wavelength.

In certain embodiments, the fluorophore comprises an emission wavelength in the range from 620 to 750 nm upon irradiation with light of an excitation wavelength.

In certain embodiments, the fluorophore comprises an emission wavelength in the range from 450 to 495 nm upon irradiation with light of an excitation wavelength.

The term “emission wavelength” refers to light having a wavelength that falls within the spectrum of light emitted by a fluorophore upon excitation

In certain embodiments, the fluorophore comprises an excitation wavelength in the blue light and/or ultraviolet range and an emission wavelength in the green light range.

In certain embodiments, the fluorophore comprises an excitation wavelength in the blue light and/or ultraviolet range and an emission wavelength in the red light range.

In certain embodiments, the fluorophore comprises an excitation wavelength in the blue light and/or ultraviolet light and an emission wavelength in the blue light range.

In certain embodiments, the polycyclic aromatic fluorophore comprises one or more cyclic aromatic groups that are directly or indirectly conjugated (delocalised electrons) with one or more other atoms.

In certain embodiments, the polycyclic aromatic fluorophore comprises a pyrene-based fluorophore. Examples of pyrene-based fluorophores include pyrene, pyranine, pentacene, perylene, terrylene and/or fluorescent derivatives of these molecules.

In certain embodiments, the polycyclic aromatic fluorophore comprises a pyrene sulphonate. In certain embodiments, the polycyclic aromatic fluorophore comprises 8-Hydroxy-1,3,6-pyrenetrisulfonic acid and/or a salt and/or a fluorescent derivative thereof. In certain embodiments, the polycyclic aromatic fluorophore comprises trisodium 8-hydroxy-1,3,6-pyrenetrisulfonate. In certain embodiments, the polycyclic aromatic fluorophore comprises a pyrene-based fluorophore comprising an emission wavelength in the range from 495 to 570 nm upon irradiation with light of an excitation wavelength.

In certain embodiments, the polycyclic aromatic fluorophore comprises a stilbene-based fluorophore. In certain embodiments, the polycyclic aromatic fluorophore comprises a substituted benzoxazolyl stilbene. In certain embodiments, the polycyclic aromatic fluorophore comprises a 4,4′-Bis(5-methyl-2-benzoxazolyl)stilbene and/or a fluorescent derivative thereof. In certain embodiments, the polycyclic aromatic fluorophore comprises a stilbene-based fluorophore comprising an emission wavelength in the range from 450 to 495 nm upon irradiation with light of an excitation wavelength.

In certain embodiments, the polycyclic aromatic fluorophore comprises a metal ion complex. In certain embodiments, the polycyclic aromatic fluorophore comprises a lanthanide or rare earth ion complex. In certain embodiments, the polycyclic aromatic fluorophore comprises a Europium ion complex. In certain embodiments, the polycyclic aromatic fluorophore comprises one or more cyclic aromatic groups directly or indirectly complexed to a metal ion. In certain embodiments, the polycyclic aromatic fluorophore comprises a metal ion complex comprising an emission wavelength in the range from 620-750 nm upon irradiation with light of an excitation wavelength.

In certain embodiments, the polycyclic aromatic fluorophore comprises a triphenylphosphine oxide. In certain embodiments, the polycyclic aromatic fluorophore comprises a triphenylphosphine oxide complexed to a metal ion. In certain embodiments, the polycyclic aromatic fluorophore comprises a triphenylphosphine oxide complexed to a lanthanide ion, such as Europium.

In certain embodiments, the polycyclic aromatic fluorophore comprises a thenoyltrifluoroacetone. In certain embodiments, the polycyclic aromatic fluorophore comprises a thenoyltrifluoroacetone complexed to a metal ion. In certain embodiments, the polycyclic aromatic fluorophore comprises a thenoyltrifluoroacetone complexed to a lanthanide ion, such as Europium.

In certain embodiments, the polycyclic aromatic fluorophore comprises a triphenylphosphine oxide and/or a thenoyltrifluoroacetone. In certain embodiments, the polycyclic aromatic fluorophore comprises a triphenylphosphine oxide and/or a thenoyltrifluoroacetone complexed to a metal ion.

In certain embodiments, the polycyclic aromatic fluorophore comprises Eu, 2-thenoyltrifluoroacetone triphenylphosphine oxide and/or a salt and/or a fluorescent derivative thereof. The structure of Eu, 2-thenoyltrifluoroacetone triphenylphosphine oxide is as follows:

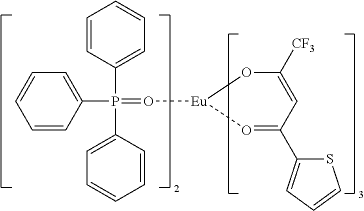

In certain embodiments, the polycyclic aromatic fluorophore comprises one or more of a pyrene-based fluorophore, a stilbene-based fluorophore, a triphenylphosphine oxide based fluorophore, a thenoyltrifluoroacetone based fluorophore and a metal ion complex.

In certain embodiments, the fluorescent composition comprises a solid composition. In certain embodiments, the fluorescent composition comprises a substantially solid composition. In certain embodiments, the fluorescent composition comprises a powder, a tablet, a dehydrate, or a lyophilised solid.

In certain embodiments, the fluorescent composition comprises a semi-solid composition. In certain embodiments, the fluorescent composition comprises a gel.

In certain embodiments, the fluorescent composition comprises a liquid composition. In certain embodiments, the fluorescent composition comprises a substantially liquid composition. In certain embodiments, the fluorescent composition comprises an aqueous composition. In certain embodiments, the fluorescent composition comprises a substantially aqueous composition.

In certain embodiments, the fluorescent composition comprises a sprayable composition. In certain embodiments, the fluorescent composition comprises an atomizable composition.

In certain embodiments, the fluorescent composition comprises a composition suitable for uptake by a plant and/or a part of a plant, such as a cut flower.

In certain embodiments, the concentration of the polycyclic aromatic fluorophore in the composition is 1.0% or less (w/w), 0.9% or less (w/w), 0.8% or less (w/w), 0.7% or less (w/w), 0.6% or less (w/w), 0.5% or less (w/w), 0.4% or less (w/w), 0.3% or less (w/w), 0.2% or less (w/w), 0.1% or less (w/w), 0.09% or less (w/w), 0.08% or less (w/w), 0.07% or less (w/w), 0.06% or less (w/w), 0.05% or less (w/w), 0.04% or less (w/w), 0.03% or less (w/w), 0.02% or less (w/w), or 0.01% or less (w/w). In certain embodiments, the concentration of the polycyclic aromatic fluorophore in the composition is one of the aforementioned concentrations expressed as weight/volume (w/v). In certain embodiments, the concentration of the polycyclic aromatic fluorophore in the composition is about one of the aforementioned concentrations.

In certain embodiments, the concentration of the polycyclic aromatic fluorophore in the composition is in the range of 0.01% to 1% (w/w), 0.01% to 0.9% (w/w), 0.01% to 0.8% (w/w), 0.01% to 0.7% (w/w), 0.01% to 0.6% (w/w), 0.01% to 0.5% (w/w), 0.01% to 0.2% (w/w), 0.01% to 0.1% (w/w), 0.01% to 0.09% (w/w), 0.01% to 0.08% (w/w), 0.01% to 0.08% (w/w), 0.01% to 0.07% (w/w), 0.01% to 0.06% (w/w), 0.01% to 0.05% (w/w), 0.05% to 1% (w/w), 0.05% to 0.9% (w/w), 0.05% to 0.8% (w/w), 0.05% to 0.7% (w/w), 0.05% to 0.6% (w/w), 0.05% to 0.5% (w/w), 0.05% to 0.2% (w/w), 0.05% to 0.1% (w/w), 0.05% to 0.09% (w/w), 0.05% to 0.08% (w/w), 0.05% to 0.07% (w/w), 0.05% to 0.06% (w/w), 0.1% to 1% (w/w), 0.1% to 0.9% (w/w), 0.1% to 0.8% (w/w), 0.1% to 0.7% (w/w), 0.1% to 0.6% (w/w), 0.1% to 0.5% (w/w), 0.1% to 0.2% (w/w), 0.2% to 1% (w/w), 0.2% to 0.9% (w/w), 0.2% to 0.8% (w/w), 0.2% to 0.7% (w/w), 0.2% to 0.6% (w/w), 0.2% to 0.5% (w/w), 0.3% to 1% (w/w), 0.3% to 0.9% (w/w), 0.3% to 0.8% (w/w), 0.3% to 0.7% (w/w), 0.3% to 0.6% (w/w), 0.3% to 0.5% (w/w), 0.4% to 1% (w/w), 0.4% to 0.9% (w/w), 0.4% to 0.8% (w/w), 0.4% to 0.7% (w/w), 0.4% to 0.6% (w/w), 0.4% to 0.5% (w/w), 0.5% to 1% (w/w), 0.5% to 0.9% (w/w), 0.5% to 0.8% (w/w), 0.5% to 0.7% (w/w), or 0.5% to 0.6% (w/w). In certain embodiments, the concentration of concentration of the polycyclic aromatic fluorophore in the composition is one of the aforementioned concentrations expressed as weight/volume (w/v). In certain embodiments, the concentration of the polycyclic aromatic fluorophore in the composition is about one of the aforementioned ranges of concentrations.

In certain embodiments, the concentration of the polycyclic aromatic fluorophore is less than 0.8% (w/w). In certain embodiments, the concentration of the polycyclic aromatic fluorophore is less than 0.8% (w/v).

In certain embodiments, the concentration of the polycyclic aromatic fluorophore is less than 0.5% (w/w). In certain embodiments, the concentration of the polycyclic aromatic fluorophore is less than 0.5% (w/v).

In certain embodiments, the concentration of the polycyclic aromatic fluorophore is 0.1% or less (w/w). In certain embodiments, the concentration of the polycyclic aromatic fluorophore is 0.1% or less (w/v). In certain embodiments, the concentration of the polycyclic aromatic fluorophore is less than 0.1% (w/w). In certain embodiments, the concentration of the polycyclic aromatic fluorophore is less than 0.1% (w/v).

In certain embodiments, the fluorescent composition further comprises a saccharide.

Certain embodiments of the present disclosure provide a fluorescent composition comprising a polycyclic aromatic fluorophore and a saccharide. Polycyclic aromatic fluorophores are as described herein.

In certain embodiments, the presence of a saccharide provides one or more further advantages: (i) the saccharide assists in the formation of droplets of a suitable size when the composition is sprayed or atomized; (ii) the saccharide assists in the formation of suitably-sized droplets on surfaces, reducing the tendency to spread over the surface, such as the surface of the petal of a flower or the surface of a leaf; (iii) the saccharide increases the amount of water retained in the droplets; and (iv) the saccharide reduces the fading of the fluorescence of a composition. Other advantages are disclosed herein.

In certain embodiments, the fluorescent composition comprises one or more saccharides. In certain embodiments, the fluorescent composition comprises a single saccharide. In certain embodiments, the fluorescent composition comprises a plurality of saccharides. In certain embodiments, the fluorescent composition comprises at least one saccharide. In certain embodiments, the fluorescent comprises at least two saccharides. In certain embodiments, the fluorescent composition comprises a combination of saccharides.

In certain embodiments, the saccharide comprises a monosaccharide, a disaccharide, a trisaccharide, an oligosaccharide a polysaccharide, or any combination of the aforementioned.

In certain embodiments, the saccharide comprises a disaccharide and/or a monosaccharide.

In certain embodiments, the saccharide comprises sucrose and/or fructose. In certain embodiments, the saccharide comprises sucrose.

In certain embodiments, the concentration of saccharide is 10% or less (w/w), 5% or less (w/w), 4% or less (w/w), 3% or less (w/w), 2% or less (w/w), 1.5% or less (w/w), 1% or less (w/w), or 0.5% or less (w/w). In certain embodiments, the concentration of saccharide is greater than 0.1% (w/w), greater than 0.2% (w/w), greater than 0.3% (w/w), greater than 0.4% (w/w); or greater than 0.5% (w/w). In certain embodiments, the concentration of saccharide is 0.5% or greater (w/w), 1% or greater (w/w), 1.5% or greater (w/w), 2% or greater, 3% or greater (w/w), 4% or greater (w/w), or 5% or greater (w/w). In certain embodiments, the concentration of saccharide is at least 0.5% (w/w), at least 1% (w/w), at least 1.5% (w/w), at least 2% (w/w), at least 3% (w/w), at least 4% (w/w), or at least 5% (w/w). In certain embodiments, the concentration of saccharide is in the range from 0.1%-10% (w/w), 0.1%-5% (w/w), 0.1%-4% (w/w), 0.1%-3% (w/w), 0.1%-2% (w/w), 0.1%-1.5% (w/w), 0.1%-1% (w/w), 0.1%-0.5% (w/w), 0.1%-0.4% (w/w), 0.1%-0.3% (w/w), 0.1%-0.2% (w/w), 0.2%-10% (w/w), 0.2%-5% (w/w), 0.2%-4% (w/w), 0.2%-3% (w/w), 0.2%-2% (w/w), 0.2%-1.5% (w/w), 0.2%-1% (w/w), 0.2%-0.5% (w/w), 0.2%-0.4% (w/w), 0.2%-0.3;% (w/w), 0.3%-10% (w/w), 0.3%-5% (w/w), 0.3%-4% (w/w), 0.3%-3% (w/w), 0.3%-2% (w/w), 0.3%-1.5% (w/w), 0.3%-1% (w/w), 0.3%-0.5% (w/w), 0.3%-0.4% (w/w), 0.4%-10% (w/w), 0.4%-5% (w/w), 0.4%-4% (w/w), 0.4%-3% (w/w), 0.4%-2% (w/w), 0.4%-1.5% (w/w), 0.4%-1% (w/w), 0.4%-0.5% (w/w), 0.5%-10% (w/w), 0.5%-5% (w/w), 0.5%-4% (w/w), 0.5%-3% (w/w), 0.5%-2% (w/w), 0.5%-1.5% (w/w) 0.5%-1% (w/w), 1%-10% (w/w), 1%-5% (w/w), 1%-4% (w/w), 1%-3% (w/w), 1%-2% (w/w), 1%-1.5% (w/w), 2%-10% (w/w), 2%-5% (w/w), or 5%-10% (w/w). In certain embodiments, the concentration of the saccharide in the composition is one of the aforementioned concentrations expressed as weight/volume (w/v). In certain embodiments, the concentration of the saccharide in the composition is about one of the aforementioned concentrations.

In certain embodiments, the concentration of the saccharide is less than 5% (w/w). In certain embodiments, the concentration of the saccharide is in the range from 0.1% to 5% (w/w). In certain embodiments, the concentration of the saccharide is in the range from 0.1 to 2% (w/w). In certain embodiments, the concentration of the saccharide in the composition is one of the aforementioned ranges expressed as weight/volume (w/v).

In certain embodiments, the concentration of the saccharide is 2% or less (w/w). In certain embodiments, the concentration of the saccharide is 2% or less (w/v).

In certain embodiments, the concentration of the saccharide in the composition is about 1.5% (w/w). In certain embodiments, the concentration of the saccharide in the composition is about 2% (w/w). In certain embodiments, the concentration of the saccharide in the composition is about 0.5% (w/w).

In certain embodiments, the fluorescent composition comprises an anti-microbial agent and/or an anti-fungal agent. In certain embodiments, the fluorescent composition comprises an anti-microbial agent and/or an anti-fungal agent that do not substantially promote wilting of a flower.

In certain embodiments, the fluorescent composition comprises one or more of a paraben (for example methylparaben), a diazolidinyl urea (for example “Germaben”), a DMDM hydantoin (for example “Glydant”), an imidazodinyl urea, a quatemium-15 (for example “Dowicil”), a benzethonium chloride (for example at a concentration of about 0.2%); benzisothiazolinine chloride, calcium benzoate, calcium tartrate, dimethyl dicarbonate (for example at a concentration of about 0.02%), natamycin (for example at a concentration of about 0.001%), 2-phenylphenol, benzoic acid and/or a salt thereof, such as sodium benzoate (for example at a concentration of about 0.1%).

In certain embodiments, the fluorescent composition comprises a fixative and/or a sealant. In certain embodiments, the fluorescent composition comprises a fixative and/or a sealant that do not substantially promote wilting of a flower when applied to a flower. Examples include Chrysal and Liquidseal, both of which are commercially available.

In certain embodiments, the fluorescent composition comprises a preservative. In certain embodiments, the fluorescent composition comprises a preservative that does not substantially promote wilting of a flower when applied to a flower. In certain embodiments, the fluorescent composition comprises one or more a paraben (for example methylparaben), a diazolidinyl urea (for example “Germaben”), a DMDM hydantoin (for example “Glydant”), an imidazodinyl urea, a quaternium-15 (for example “Dowicil”), a benzethonium chloride (for example at a concentration of about 0.2%); benzisothiazolinine chloride; calcium benzoate, calcium tartrate, dimethyl dicarbonate (for example at a concentration of about 0.02%), natamycin (for example at a concentration of about 0.001%), 2-phenylphenol, benzoic acid and/or a salt thereof, such as sodium benzoate (for example at a concentration of about 0.1%).

In certain embodiments, the fluorescent composition comprises an acid. In certain embodiments, the fluorescent composition comprises a weak acid, being an acid that dissociates incompletely. In certain embodiments, the fluorescent composition comprises an organic acid. In certain embodiments, the fluorescent composition comprises a weak organic acid. In certain embodiments, the fluorescent composition comprises citric acid and/or acetic acid. In certain embodiments, an acid may function as an antimicrobial agent.

In certain embodiments, the fluorescent composition comprises a base. In certain embodiments, the fluorescent composition comprises a weak base, being a base that does not ionize fully in solution. In certain embodiments, the fluorescent composition comprises an organic base and/or an inorganic base. In certain embodiments, the fluorescent composition comprises a carbonate and/or bicarbonate.

In certain embodiments, the fluorescent formulation comprises about 0.1% bicarbonate.

In certain embodiments, the fluorescent composition comprises a buffer. In certain embodiments, the fluorescent composition comprises an organic buffer and/or an inorganic buffer. In certain embodiments, the fluorescent composition comprises a carbonate and/or bicarbonate.

In certain embodiments, the fluorescent composition comprises one or more of an acid, a base and a buffering agent.

In certain embodiments, the fluorescent composition comprises a substantially aqueous composition. In certain embodiments, the fluorescent composition comprises an aqueous composition with no additional solvents. In certain embodiments, the fluorescent composition comprises an aqueous composition and one or more additional solvents, such as ethylene glycol.

In certain embodiments, the fluorescent composition comprises a weakly acid pH. In certain embodiments, the fluorescent composition comprises a substantially neutral pH. In certain embodiments, the fluorescent composition comprises a weakly basic pH. Methods for adjusting pH are known.

In certain embodiments, the pH of the composition is greater than 3.0, greater than 3.5, greater than 4.0, greater than 4.4, greater than 4.9, greater than 5.0, greater than 6.0, greater than 6.8, greater than 7.0, or greater than 7.5, greater than 8.0, or greater than 8.5. In certain embodiments, the pH of the composition is at least 3.0, at least 3.5, at least 4.0, at least 4.4, at least 4.9, at least 5.0, greater than 6.0, at least 6.8, at least 7.0, at least 7.5, at least 8.0, at least 8.5, or at least 9.0. In certain embodiments, the pH of the composition is about one of the aforementioned pHs.

In certain embodiments, the pH of the composition is in the range from 3.0 to 9.0. 3.0 to 8.0, 3.0 to 7.5, 3.0 to 7.0, 3.0 to 6.6, 3.0 to 6.0, 3.0 to 5.5, 3.0 to 5.0, 3.0 to 4.5, 3.0 to 4.4, 3.5 to 9.0. 3.5 to 8.0, 3.5 to 7.5, 3.5 to 7.0, 3.5 to 6.6, 3.5 to 6.0, 3.5 to 5.5, 3.5 to 5.0, 3.5 to 4.5, 3.5 to 4.4, 4.0 to 9.0. 4.0 to 8.0, 4.0 to 7.5, 4.0 to 7.0, 4.0 to 6.6, 4.0 to 6.0, 4.0 to 5.5, 4.0 to 5.0, 4.0 to 4.5, 4.0 to 4.4, 4.4 to 9.0, 4.4 to 8.0, 4.4 to 7.5, 4.4 to 7.0, 4.4 to 6.6, 4.4 to 6.0, 4.4 to 5.5, 4.4 to 5.0, 4.4 to 4.5, 5.0 to 9.0, 5.0 to 8.0, 5.0 to 7.5, 5.0 to 7.0, 5.0 to 6.6, 5.0 to 6.0, 5.0 to 5.5, 5.5 to 9.0, 5.5 to 8.0, 5.5 to 7.5, 5.5 to 7.0, 5.5 to 6.6, 5.5 to 6.0, 6.0 to 9.0, 6.0 to 8.0, 6.0 to 7.5, 6.0 to 7.0, 6.0 to 6.6, 6.5 to 9.0, 6.5 to 8.0, 6.5 to 7.5, 6.5 to 7.0, 6.5 to 6.6, 7.0 to 9.0, 7.0 to 8.0, 7.0 to 7.5, or 7.5 to 9.0, or 7.5 to 8.0. In certain embodiments, the pH of the composition is about one of the aforementioned ranges of pHs.

In certain embodiments, the fluorescent composition comprises a pH of 3.0 or greater. In certain embodiments, the fluorescent composition comprises a pH of 3.5 or greater. In certain embodiments, the fluorescent composition comprises a pH of 4.4 or greater.

In certain embodiments, the fluorescent composition comprises a pH of about 7.5.

In certain embodiments, the fluorescent composition comprises an organic solvent to assist with dissolution of a fluorophore which is hydrophobic and/or to assist with slowing the precipitation of a fluorophore that is hydrophobic once in solution. For example, ethylene glycol is suitable for dissolving a hydrophobic fluorophore and/or for slowing precipitation of the fluorophore.

In certain embodiments, the fluorescent composition comprises an ethylene glycol.

In certain embodiments, the fluorescent composition comprises one or more of anti-microbial agent, an anti-fungal agent, a fixative, a sealant, a preservative, an acid, a base, a buffering agent and an organic solvent.

In certain embodiments, the fluorescent composition is treated to reduce micro-organisms. Examples of such treatments include one or more of filtering, irradiation and heat and/or pressure sterilisation. In certain embodiments, the fluorescent composition is sterilised. In certain embodiments, the fluorescent composition is heat and/or pressure sterilised, such as by autoclaving.

In certain embodiments, the fluorescent composition is a sprayable composition.

Examples of fluorescent compositions suitable for spraying onto flowers and/or plants are as follows:

GREEN: 0.1% pyranine (8-hydroxypyrene-1,3,6-trisulfonic acid)
1.5% sugar
0.1% sodium bicarbonate
0.1% sodium benzoate
pH 7.5
Aqueous Formulation

RED: 0.07% Eu, 2-thenoyltrifluoroacetone triphenylphosphine oxide.
2.0% sugar
0.1% sodium bicarbonate
0.1% sodium benzoate
pH 7.5
The fluorescent agent is dissolved in ethylene glycol at 0.7%, to which is then added water to give the final concentration of 0.07%.

BLUE: 0.03% 4,4′-bis(2-benzoxazolyl)stilbene.
1.5% sugar
0.1% sodium bicarbonate
0.1% sodium benzoate
pH 7.5
The fluorescent agent is dissolved in ethylene glycol at 0.3%, to which is then added water to give the final concentration of 0.03%.

In certain embodiments, the fluorescent composition is an atomizable composition.

In certain embodiments, the fluorescent composition when atomised and sprayed onto a waxy surface, the spray forms droplets on the surface. In certain embodiments, the fluorescent composition, when atomised and sprayed onto a surface of a flower, the spray forms droplets on the surface of the flower.

In certain embodiments, the fluorescent composition is a composition for marking an object.

Certain embodiments of the present disclosure provide the use of a fluorescent composition as described herein for marking an object.

Certain embodiments of the present disclosure provide the use of a polycyclic aromatic fluorophore as described herein in the preparation of a composition for marking an object.

Certain embodiments of the present disclosure provide use of a fluorescent composition comprising a polycyclic aromatic fluorophore for marking an object.

Certain embodiments of the present disclosure provide the use of a polycyclic aromatic fluorophore and a saccharide as described herein in the preparation of a composition for marking an object.

The term “marking” and variants such as “mark” or “marked” refer to a fluorescent agent being added to a product, and include for example a fluorescent agent being applied to a surface of a product and/or a fluorescent being incorporated into a product. For example, a fluorescent agent may be incorporated into all or part of a plant by uptake of the fluorescent agent by the plant.

Certain embodiments of the present disclosure provide a fluorescent marking composition, the composition comprising a polycyclic aromatic fluorophore. Polycyclic aromatic fluorophores are as described herein.

Certain embodiments of the present disclosure provide a fluorescent marking composition, the composition comprising a polycyclic aromatic fluorophore and a saccharide.

Certain embodiments of the present disclosure provide an object comprising a fluorescent composition as described herein.

Certain embodiments of the present disclosure provide an object comprising a fluorescent composition, the composition comprising a polycyclic aromatic fluorophore. Polycyclic aromatic fluorophores are as described herein.

Certain embodiments of the present disclosure provide an object comprising a fluorescent composition, the composition comprising a polycyclic aromatic fluorophore and a saccharide.

In certain embodiments, the object comprises a manufactured product or an article, such as a novelty product. Examples of products include toys, clothing or fashion accessories, and paints.

In certain embodiments, the object comprises an organism and/or a part of an organism. In certain embodiments, the object comprises an ornamental organism and/or part of an ornamental organism. In certain embodiments, the object comprises all or part of one or more plants. In certain embodiments, the object comprises all or part of one or more flowers.

In certain embodiments, the organism is one or more ornamental organisms. In certain embodiments, the ornamental organism comprises one or more plants. Other types of ornamental organisms are also contemplated, such as ornamental fungi. The term “ornamental” refers to an organism that has one or more aesthetic characteristics.

The term “plant” includes vascular angiosperm and gymnosperm plants; non-vascular or bryophyte plants such as mosses, liverworts and hornworts; ferns and other non-flowering, non-bryophyte plants (for example Selaginella); algae including terrestrial, aquatic and marine algae; and living whole plants, as well as cut, harvested or dried plant material such as cut or harvested foliage, flowers, seeds or other plant parts. The term includes live plants and/or cut plants.

In certain embodiments, the object comprises one or more flowers, such as one or more cut flowers and/or one or more live flowers. In certain embodiments, the object comprises one or more plants, such as one or more plants with one or more variegated leaves. For example, in certain embodiments the composition is applied to and/or introduced into one or more plants and/or one or more flowers in an arrangement, such as a vase.

In certain embodiments, there is provided an arrangement of one or more plants comprising the fluorescent composition as described herein. In certain embodiments, there is provided an arrangement of one or more flowers comprising the fluorescent composition as described herein. For example, an arrangement of one or more plants and/or flowers may comprise a fluorescent composition as described herein applied externally and/or introduced by uptake to the one or more plants and/or flowers.

In certain embodiments there is provided a vase comprising one or more plants and/or flowers comprising the fluorescent composition as described herein. For example, a vase comprising one or more plants and/or flowers may comprise a fluorescent composition as described herein applied externally and/or introduced by uptake to the one or more plants and/or flowers.

In certain embodiments, there is provided a corsage comprising one or more plants and/or flowers comprising the fluorescent composition as described herein. For example, a corsage comprising one or more plants and/or flowers may comprise a fluorescent composition as described herein applied externally and/or introduced by uptake to the one or more plants and/or flowers.

In certain embodiments there is provided a lapel flower or lapel floral arrangement comprising the fluorescent composition as described herein. For example, a lapel flower or lapel floral arrangement comprising one or more plants and/or flowers may comprise a fluorescent composition as described herein applied externally and/or introduced by uptake to the one or more plants and/or flowers.

In certain embodiments, the object comprises one or more plants selected from the list consisting of: rose (Rosa×hybrida), chrysanthemum (Dendranthema grandiflora), lily (Lilium spp.), gerbera (Gerbera×hybrida), tulip (Tuilpa sp.), carnation (Dianthus caryophyllus), alstromeria (Alstromeria sp.), gladiolus (Gladiolus spp.), anthurium (Anthurium andraeanum), Antirrhinum spp. (sometimes referred to as snapdragons), gypsophila (Gypsophila paniculata, Gypsophila elegans), orchids such as Phalaenopsis, Cymbidium and Dendrobium spp, and Paphiopedilum (lady's slipper) orchid, cyclamen (Cyclamen persicum), azalea (Rhododendron sp.), African violet (Saintpaulia ionantha), petunia (Petunia×hybrida), impatiens (Impatiens sp.), begonia (Begonia sp.), Physalis, geranium (Pelargonium sp.), pansy (Viola sp.), marigold (Tagetes erecta), Dracaena, Ficus, Spathiphyllum, Peperomia, Sanseveria and a number of fern, conifer, cacti, and palm species. In certain embodiments, the plant comprises one or more flowers from the aforementioned plants. The aforementioned plants may be cut plants and/or live plants.

In certain embodiments, the object is selected from one or more of one or more roses, one or more orchids, one or more carnations, one or more lilies, one or more lisianthus, one or more jasmine, one or more baby's breath (gypsophila), one or more chrysanthemum, one or more white poppies, one or more bellflowers, one or more fuchsia, one or more gerberas, and one or more daisies. The aforementioned flowers are particularly suitable for illumination in use in an arrangement, in a corsage, a lapel flower, a fashion accessory, or a vase. The aforementioned flowers may be cut flowers and/or live flowers.

In certain embodiments, the object is a single flower, such a rose. In certain embodiments, the object is a collection of flowers, such as a bouquet of flowers. In certain embodiments, the plants have flowers with one or more white flowers, one or more pink flowers, one or more red flowers, one or more purple flowers, one or more blue flowers, one or more yellow flowers, or combinations of these colours. Flowers with other colours of flowers, or combinations of colours, are contemplated. In certain embodiments, the flowers comprise petals of a lighter hue. The aforementioned flowers may be cut flowers and/or live flowers.

Certain embodiments provide an arrangement of one or more plants and/or one or more flowers comprising the fluorescent composition as described herein.

In certain embodiments, the object comprises one or more flowers. In certain embodiments, the object comprises one or more cut flowers and/or live flowers.

In certain embodiments, the object comprises a vase comprising one or more flowers, such as one or more live flowers and/or one or more cut flowers.

Certain embodiments provide a vase comprising one or more flowers, the one or more flowers comprising a fluorescent composition as described herein. In certain embodiments, the object comprises a vase comprising one or more flowers and/or one or more plants.

Certain embodiments provide a corsage comprising one or more flowers, the one or more flowers comprising a fluorescent composition as described herein.

In certain embodiments, the object comprises a lapel flower. Certain embodiments provide a lapel flower comprising one or more flowers, the one or more flowers comprising a fluorescent composition as described herein.

In certain embodiments, the fluorescent composition is applied to a surface of the object. In certain embodiments, the fluorescent composition is introduced into an object.

Certain embodiments of the present disclosure provide an object comprising a fluorescent composition applied to a surface of the object, the fluorescent composition comprising a polycyclic aromatic fluorophore.

Certain embodiments of the present disclosure provide an object comprising a fluorescent composition applied to a surface of the object, the fluorescent composition comprising a polycyclic aromatic fluorophore and a saccharide.

Certain embodiments of the present disclosure provide an arrangement of one or more flowers and/or plants, the one or more flowers and/or plants comprising a fluorescent composition as described herein applied to a surface of the one or more flowers and/or plants.

Certain embodiments of the present disclosure provide an arrangement of one or more flowers and/or plants, the one or more flowers and/or plants comprising a fluorescent composition comprising a polycyclic aromatic fluorophore, the fluorescent composition applied to a surface of the one or more flowers and/or plants.

In certain embodiments, the object comprises a fluorescent composition as described herein introduced and/or incorporated into the object. For example, the fluorescent composition may be incorporated into all or part of an object during the manufacturing process.

In certain embodiments, the object comprises a plant and/or a part of a plant comprising the fluorescent composition as described herein introduced into the plant (or part of the plant) by uptake.

Certain embodiments of the present disclosure provide the use of a fluorescent composition as described herein for uptake by a plant and/or flower.

Certain embodiments of the present disclosure provide the use of a polycyclic aromatic fluorophore as described herein in the preparation of a composition for uptake by a plant and/or flower.

Certain embodiments of the present disclosure provide use of a fluorescent composition comprising a polycyclic aromatic fluorophore for uptake by a plant and/or flower.

Certain embodiments of the present disclosure provide the use of a polycyclic aromatic fluorophore and a saccharide as described herein in the preparation of a composition for uptake by a plant and/or flower.

In certain embodiments, the object is a plant and/or a part thereof comprising a fluorescent composition, the fluorescent composition introduced into the plant or the part thereof by uptake of the fluorescent composition by the plant or the part thereof. In certain embodiments, the plant and/or part thereof comprises one or more flowers.

In certain embodiments, the object is a plant and/or a part thereof comprising a fluorophore, the fluorophore introduced into the plant or the part thereof by uptake of a fluorescent composition as described herein by the plant or the part thereof. In certain embodiments, the plant and/or part thereof comprises one or more flowers.

Certain embodiments of the present disclosure provide a plant and/or a part thereof comprising a fluorophore introduced into the plant and/or the part thereof by uptake of a fluorescent composition by the plant or the part thereof, the fluorescent composition comprising a polycyclic aromatic fluorophore. Polycyclic aromatic fluorophores are as described herein.

Certain embodiments of the present disclosure provide a plant and/or a part thereof comprising a fluorophore introduced into the plant and/or the part thereof by uptake of a fluorescent composition by the plant or the part thereof, the fluorescent composition comprising a polycyclic aromatic fluorophore and a saccharide.

Certain embodiments of the present disclosure provide an arrangement of one or more flowers and/or plants, the one or more flowers and/or plants comprising a fluorescent composition as described herein being taken up by the one or more flowers and/or one plants.

Certain embodiments of the present disclosure provide an arrangement of one or more flowers and/or plants, the one or more flowers and/or plants comprising a fluorescent composition comprising a polycyclic aromatic fluorophore, the fluorescent composition being taken up by the one or more flowers and/or plants.

Methods for introducing exogenous agents into a plant (and/or a part thereof) by uptake are known. In certain embodiments, the methods as described herein comprise introducing a fluorescent composition as described herein into the plant (and/or a part thereof) by uptake. In certain embodiments, the methods as described herein comprise introducing a polycyclic fluorophore as described herein into the plant (and/or a part thereof) by uptake.

In certain embodiments, a live plant is placed in an environment comprising the fluorescent composition as described herein. In certain embodiments, the cut end of stems of flowers or the cut ends of part of a plant are placed directly in a solution comprising a fluorescent composition as described herein.

Certain embodiments of the present disclosure provide a method of producing a fluorescent plant (and/or a part thereof) by introducing a fluorophore into the plant (and/or a part thereof) by uptake of a fluorescent composition as described herein. Certain embodiments of the present disclosure provide a plant and/or part thereof produced by the method as described herein.

Certain embodiments of the present disclosure provide a method of producing a fluorescent plant (and/or a part thereof), the method comprising introducing a fluorophore into the plant (and/or a part thereof) by uptake of the plant and/or the part thereof of a fluorescent composition comprising a polycyclic aromatic fluorophore. Polycyclic aromatic fluorophores are as described herein.

Certain embodiments of the present disclosure provide a method of producing a fluorescent plant (and/or a part thereof), the method comprising introducing a fluorophore into the plant (and/or a part thereof) by uptake of the plant and/or the part thereof of a fluorescent composition comprising a polycyclic aromatic fluorophore and a saccharide.

Certain embodiments of the present disclosure provide a method of producing a fluorescent plant and/or a part thereof, the method comprising introducing a fluorescent composition as described herein into the plant and/or a part thereof by uptake.

Certain embodiments of the present disclosure provide a method of producing a fluorescent plant and/or or a part thereof, the method comprising introducing a fluorescent composition comprising a polycyclic aromatic fluorophore into the plant and/or a part thereof by uptake.

Certain embodiments of the present disclosure provide a method of producing a fluorescent plant and/or or a part thereof, the method comprising introducing a polycyclic aromatic fluorophore into the plant and/or a part thereof by uptake. Polycyclic aromatic fluorophores are as described herein.

In certain embodiments, the fluorescent composition as described herein is applied to an object. In certain embodiments, the fluorescent composition is applied to a surface of an object. In certain embodiments, the fluorescent composition is applied to an external surface of an object, such as a surface of flower and/or a plant. Methods for applying a composition to an object are known and include, for example, spraying, painting, dipping, immersion, printing, bombardment, or impregnation.

In certain embodiments, a spraying device or an atomizing device is used to apply the fluorescent composition to an object. Spraying devices and atomizing devices are known, and include for example, manually actuated spraying devices and atomizers, pump action spraying devices and atomizers, and pressure spraying devices and atomizers such as a spray cans and spray bottles, including such devices utilising a propellant.

Certain embodiments of the present disclosure provide a spraying device or an atomizing device comprising a fluorescent composition as described herein.

Certain embodiments of the present disclosure provide a spraying device comprising a fluorescent composition, wherein the fluorescent composition comprises a polycyclic aromatic fluorophore.

Certain embodiments of the present disclosure provide a spraying device comprising a fluorescent composition, wherein the fluorescent composition comprises a polycyclic aromatic fluorophore and a saccharide.

Certain embodiments of the present disclosure provide an atomizing device comprising a fluorescent composition, wherein the fluorescent composition comprises a polycyclic aromatic fluorophore.

Certain embodiments of the present disclosure provide an atomizing device comprising a fluorescent composition, wherein the fluorescent composition comprises a polycyclic aromatic fluorophore and a saccharide.

In certain embodiments, the fluorescence of the fluorescent composition is viewed by illuminating the fluorescent composition with light comprising light of a wavelength capable of excitation of the polycyclic aromatic fluorophore. For example, in the case of a fluorophore that has an excitation wavelength in the blue or near ultraviolet ranges, the fluorescence may be viewed under black light conditions.

In certain embodiments, the fluorescence of the fluorescent composition is viewed by illuminating the fluorescent composition with a device emitting light comprising light of a wavelength capable of excitation of the fluorophore. For example, in the case of a fluorophore that has an excitation wavelength in the blue and/or near ultraviolet range, the fluorescence may be viewed by illuminating the fluorescent composition with a blue/UV torch or other type of device that emits light of an excitation wavelength. Such devices for illumination are known and commercially available.

Certain embodiments of the present disclosure provide a combination product for using a fluorescent composition as described herein. Such products may be used, for example, to mark an object and/or to improve the aesthetic appeal of an object.

Certain embodiments of the present disclosure provide a combination product comprising a fluorescent composition as described herein.

In certain embodiments, the combination product comprises a device for applying the fluorescent composition to an object, as described herein. In certain embodiments, the combination product comprises a device for applying the fluorescent composition to the surface of an object, such as a spraying device or an atomizing device.

Certain embodiments of the present disclosure provide a combination product, the combination product comprising the following components: a fluorescent composition comprising a polycyclic aromatic fluorophore; and an atomizing device.

Certain embodiments of the present disclosure provide a combination product, the combination product comprising the following components: a fluorescent composition comprising a polycyclic aromatic fluorophore and a saccharide; and an atomizing device.

In certain embodiments, the combination product comprises one of more of an acid, base, a solvent and a buffering agent, as described herein.

Certain embodiments of the present disclosure provide a kit comprising a fluorescent composition as described herein. Such kits may be used, for example, to mark an object and/or to improve the aesthetic appeal of an object.

Certain embodiments of the present disclosure provide a kit comprising a fluorescent composition comprising a polycyclic aromatic fluorophore.

Certain embodiments of the present disclosure provide a kit for marking an object as described herein.

Certain embodiments of the present disclosure provide a kit comprising a fluorescent composition, the fluorescent composition comprising a polycyclic aromatic fluorophore. In certain embodiments, the composition is in a solid form or a liquid form, as described herein.

Certain embodiments of the present disclosure provide a kit comprising a fluorescent composition, the fluorescent composition comprising a polycyclic aromatic fluorophore and a saccharide.

In certain embodiments, the kit comprises a spraying device and/or an atomizing device as described herein.

In certain embodiments, the kit comprises a device for illuminating an object comprising the fluorescent composition. Devices for illuminating an object comprising the fluorescent composition are described herein.

In certain embodiments, the kit comprises one or more of an acid, a base, a solvent, and a buffering agent, as described herein.

Certain embodiments of the present disclosure provide a kit for performing a method as described herein.

In certain embodiments, the kit comprises one or more reagents as described herein and/or instructions. In certain embodiments the instructions comprise one or more of instructions for preparing a fluorescent composition as described herein, instructions for use of a fluorescent composition in a spraying device or atomizing device as described herein, instructions for applying a fluorescent composition to an object as described herein, instructions for introducing a fluorophore into a plant by uptake as described herein, instructions for producing a fluorescent plant as described herein, and instructions for viewing fluorescence as described herein.

In certain embodiments, the kit comprises instructions for applying the fluorescent composition to a surface of an object, such as plants and/or flowers. In certain embodiments, the kit comprises instructions for introducing a fluorophore into a plant by uptake.

Certain embodiments of the present disclosure provide a method of applying a fluorescent agent to an object, the method comprising applying a fluorescent composition as described herein.

Certain embodiments of the present disclosure provide a method of applying a fluorescent agent to an object, the method comprising spraying the object with a fluorescent composition as described herein.

Certain embodiments of the present disclosure provide a method of applying a fluorescent agent to a surface of an object, the method comprising spraying the object with a fluorescent composition as described herein.

Certain embodiments of the present disclosure provide a method of marking an object.

Certain embodiments of the present disclosure provide a method of marking an object, the method comprising applying a fluorescent composition to the object as described herein.

Certain embodiments of the present disclosure provide a method of marking an object, the method comprising spraying the object with a fluorescent composition as described herein.

Certain embodiments of the present disclosure provide a method of marking an object, the method comprising introducing a fluorescent composition into the object as described herein.

Certain embodiments of the present disclosure provide a method of improving the aesthetic quality of an object

Certain embodiments of the present disclosure provide a method of improving the aesthetic quality of an object, the method comprising: applying a fluorescent composition to a surface of the object, the fluorescent composition comprising a polycyclic aromatic fluorophore; and illuminating the object with light of an excitation wavelength to excite the polycyclic aromatic fluorophore in the fluorescent composition.

Certain embodiments of the present disclosure provide a method of improving the aesthetic quality of an object, the method comprising: introducing a fluorescent composition into the object, the fluorescent composition comprising a polycyclic aromatic fluorophore; and illuminating the object with light of an excitation wavelength to excite the polycyclic aromatic fluorophore in the fluorescent composition.

Certain embodiments of the present disclosure provide a method of illuminating an object comprising a fluorescent composition, as described herein.

In certain embodiments, the method comprises applying a fluorescent composition to a surface of the object, as described herein. In certain embodiments, the method comprises introducing a fluorophore into a plant, as described herein.

In certain embodiments, the method comprises illuminating the object with light of an excitation wavelength to excite the polycyclic aromatic fluorophore in the fluorescent composition, as described herein.

Certain embodiments of the present disclosure provide a method of illuminating an object, the method comprising: applying a fluorescent composition to a surface of the object, the fluorescent composition comprising a polycyclic aromatic fluorophore; and illuminating the object with light of an excitation wavelength to excite the polycyclic aromatic fluorophore in the fluorescent composition.

Certain embodiments of the present disclosure provide a method of illuminating an object, the method comprising: applying a fluorescent composition to a surface of the object, the fluorescent composition comprising a polycyclic aromatic fluorophore and a saccharide; and illuminating the object with light of an excitation wavelength to excite the polycyclic aromatic fluorophore in the fluorescent composition.

Certain embodiments of the present disclosure provide a method of illuminating an object, the method comprising: introducing a fluorescent into the object, the fluorescent composition comprising a polycyclic aromatic fluorophore; and illuminating the object with light of an excitation wavelength to excite the polycyclic aromatic fluorophore in the fluorescent composition.

### Example 1

### Fluorescent Agents

Different types of fluorophores were tested to see the effect on flowers and to observe the fluorescence emitted when on the flowers.

The following fluorophores were tested at the following concentrations: Xanthene based fluorophores:

(i) 0.5% (w/v) Eosin Y (ii) 0.5% (w/v) Rhodamine 6G;
(iii) 0.5% (w/v) Fluorescein; Polycyclic Aromatic Hydrocarbon based Fluorophores:
(i) 0.05% (w/v) 8-Hydroxypyrene-1,3,6-trisulfonic acid trisodium salt (also known as Pyranine (an arylsulphonate flurophore) or “Solvent Green”).

The above solutions were prepared by adding the fluorophore to tap water, in order to test the properties of the fluorophores in a non-sterilised, non-purified aqueous solution. The solutions were placed into a spray device for application to the surface of the relevant plants.

Solutions were sprayed onto chrysanthemums and roses and allowed to dry overnight at room temperature. Solutions of Xanthene based fluorophores (Eosin Y, Fluorescein and Rhodamine 6G) were rejected, since these solutions caused wilting of flowers and reduced vase life.

A solution of 0.05% (w/v) Fluorescent brightener 28 (also known as Calcofluor White M2R) was also tested. Fluorescent brightener 28 was rejected since it emitted blue fluorescent light which was not very effective, when excited by UVA light.

It was found that 0.05% (w/v) pyranine did not affect the vase life of flower and provided a bright green fluorescence, when excited by UVA light.

It was concluded that fluorophores providing a strong emission in the green light spectrum were effective. It was further concluded that Xanthene-based fluorophores promoted wilting and reduced vase life, while polycyclic aromatic fluorophores (typified by pyranine) did not promote wilting and/or reduce vase life.

### Example 2

### Pyranine Emission is pH Dependent

Flowers were sprayed with 0.05% (w/v) pyranine solution at a pH of 7.0. The liquid was allowed to dry overnight at room temperature. Another solution containing 0.05% (w/v) pyranine was prepared and the pH of the solution altered to 3.0 using acetic acid solution. This solution was also sprayed on the flowers and allowed to dry overnight at room temperature.

It was observed that flowers sprayed with 0.05% (w/v) pyranine (pH of 7.0) emitted fluorescent green spots when excited by UVA light, while 0.05% (w/v) pyranine at pH 3.0 emitted a fluorescent blue colour, when excited by UV light. The pH of the solution was therefore found to affect the colour of the fluorescence emitted, when excited by UVA light. This result further demonstrated the ability of pyranine as a fluorophore whose fluorescence colour can be altered and maintained when sprayed onto a surface by alteration of pH.

### Example 3

### Effect of a Polyethylene Glycol on Fluorescence

To determine whether a polyethylene glycol may enhance fluorescence of a fluorophore, the effect of polyethylene glycol 1500 was tested on fluorescein applied to flowers.

Polyethylene glycol at concentrations of 0.3% (w/v), 0.5% (w/v), 1% (w/v), 2% (w/v) concentration was tested. It was observed that the pH of the solution was lowered to 3.36-2.64 when 0.3% (w/v) to 5% (w/v) polyethylene glycol 1500 was added to the solution. Flowers were sprayed with the solution containing the respective concentrations of polyethylene glycol 1500 and allowed to dry overnight. A negative control with no spray was also used to monitor the experiment to determine vase life.

It was found that all flowers sprayed with the fluorophore solution containing polyethylene glycol emitted blue colour fluorescence, while the flowers sprayed with only the fluorophore solution emitted a green fluorescence when excited by UV light.

### Example 4

### Altering pH with Acetic Acid as Compared to Citric Acid

Lemon juice (a source of citric acid) and vinegar (a source of acetic acid) were added to the fluorophore solution to decrease the pH of the solution so as to obtain blue fluorescence. The effect of lemon juice and vinegar was compared with each other.

A solution containing 0.1% (w/v) of pyranine was prepared and lemon juice was added to adjust the pH of the solution to 3.0. A similar fluorophore solution, containing 0.1% (w/v) of pyranine was prepared and the pH of the solution was also adjusted to 3.0. The solutions were sprayed on individual flowers and were allowed to dry overnight at room temperature. A negative control with no spray was also used to monitor the experiment to determine vase life.

Flowers sprayed with fluorophore solution containing lemon juice and flowers sprayed with fluorophore solution containing vinegar emitted blue fluorescent spots, when excited by UV light. It was observed that the colour of the fluorescent spots, on flowers sprayed with fluorophore solution containing vinegar, changed back to a green colour after 2 days. This effect may be attributable to acetic acid vaporising with time, hence altering the pH of the fluorophore and changing the colour of the fluorescence emitted.

### Example 5

### Concentration of Pyranine

The effect of different pyranine concentrations was tested to determine the relationship between concentration and fluorescence, and to determine if a higher concentration of the fluorophore affected the vase life of the flower.

Concentrations of 0.05% (w/v), 0.1% (w/v), 0.2% (w/v) and 0.5% (w/v) pyranine were tested. Solutions were prepared with the respective concentrations of fluorophore using tap water. The solutions were applied on individual flowers and were allowed to dry overnight. A negative control with no spray was also used to monitor the experiment to determine vase life.

The amount of fluorescence emitted by the flowers sprayed with various concentrations of pyranine solutions appeared to fade with increasing concentration of pyranine when excited by UV light. It was observed that with 0.2% (w/v) and 0.5% (w/v) concentration of pyranine the colour of the droplets turned from fluorescent green to non-fluorescent yellow. It was also found that the various concentrations of pyranine did not affect vase life. Hence, 0.1% (w/v) of pyranine was therefore selected as a suitable concentration for all future experiments.

### Example 6

### Effect of Sealants

Different sealants were tested to see if they would preserve the fluorophore on the flower and to test whether they prevented the fluorophore from being rubbed away. Chrysal Professional Glory and Pokon Leaf Shine were tested for this purpose.

Flowers were sprayed with 0.1% (w/v) pyranine solution and allowed to dry for 2 hours at room temperature. It was then sprayed with Chrysal Professional Glory and allowed to dry for 2 hours. Another bunch of flowers was sprayed with the pyranine solution, and after drying Leaf Shine spray applied. A negative control with no spray was also used to monitor the experiment to determine vase life.

In both cases, the flower wilted and the vase-life of the flower was found to be reduced drastically. In both cases the fluorophore rubbed off on the hand and it did not preserve the fluorophore, hence both products were not considered further.

In a further experiment, the ability to protect the fluorophore from being rubbed off was tested by making up pyranine with Chrysal Professional Glory solution.

The solution from Chrysal Professional Glory was mixed with pyranine to give a 0.1% (w/v) solution. This solution was sprayed on the flowers and was allowed to dry overnight. A negative control with no spray was also used to monitor the experiment to determine vase life.

The colour of the fluorescence emitted by the fluorophore was yellowish green, when excited by UV light. The fluorophore rubbed off on the hand and was not preserved on the flower. Hence Chrysal Professional Glory was not used further.

### Example 7

### pH Dependence of Pyranine Fluorescence

To further investigate the pH dependence of pyranine fluorescence, the various colours emitted by the fluorophore was tested by altering the pH of the solution using lemon juice (a source of citric acid).

The solution was made with 0.1% pyranine (w/v), 5% (w/v) sugar and the pH of the solution was altered from 6.84 to 6.00, 4.95, 4.46, 4.01, 3.69, 3.48, 3.01 using lemon juice. The fluorophore solutions with the different pH was made and sprayed onto flowers, which were allowed to dry overnight. A negative control with no spray was also used to monitor the experiment to determine vase life.

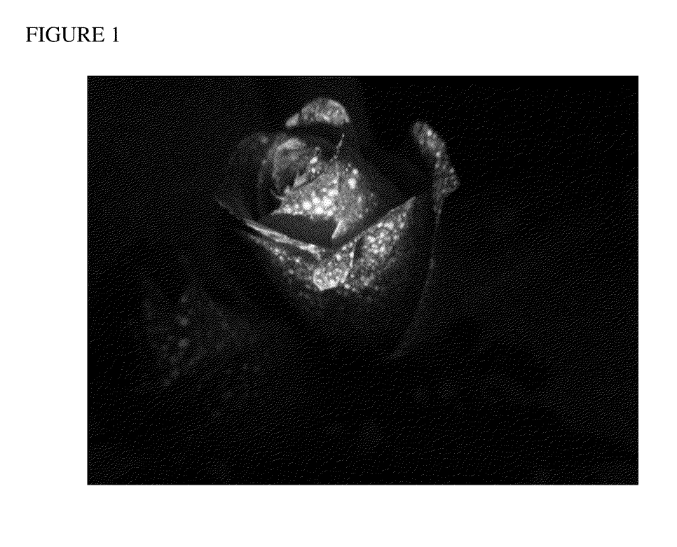

Fluorescent Green spots were observed between a pH of 6.84 to 4.46 and fluorescent blue spots were observed between a pH of 4.46 to 3.01, when excited by UV light. FIG. 1A shows flowers sprayed with 0.1% (w/v) pyranine solution at a pH of 3.0 adjusted, when excited by UV light. FIG. 1B shows flowers sprayed with 0.1% (w/v) pyranine solution at a pH of 6.84, when excited by UV light.

### Example 8

### Effect of Time of UV Exposure

The effect of long term exposure of UV light was tested on flowers sprayed with fluorophore

Flowers were sprayed with 0.1% (w/v) pyranine solution. The flowers were allowed to dry overnight. A UV torch Optimax™ 365, having an intensity of 50,000 μW/cm2 was shone on the sprayed flowers. It was observed for 1, 2, 4, and 6 hours.

There was no visible change in the colour of fluorescence emitted on the flowers even after 6 hours of continuous UV exposure. Hence it was concluded that the fluorescence emitted by the pyranine 0.1% (w/v) solution sprayed on the flower is consistent even after long hours of UV exposure.

### Example 9

### Effect of Sugars on Droplet Formation

The effect of different types of sugar on the droplet formation of the pyranine fluorophore when sprayed on the flowers was tested.

A pyranine solution at 0.1% (w/v) was prepared with 5% sucrose. In addition, a 0.1% (w/v) pyranine solution with 5% glucose was also prepared. The solutions were sprayed onto flowers and allowed to dry overnight. A 0.1% (w/v) pyranine solution was also prepared where no sugar was added. A negative control with no spray was also used to monitor the experiment to determine vase life.

It was observed that 0.1% (w/v) pyranine solution with 5% sucrose gave mist-like droplet formation, where the fluorescent green droplet is distributed all over the petal, as seen when excited by UV light. Glucose gave a similar type of mist like fluorescent green droplet distribution. With no sugar, the fluorophore spread throughout the surface of petal of the flower and did not form fine droplets, in part due to the petal of the flower being waxy and no sugar in solution to induce droplet formation of the pyranine fluorophore.

These and other studies demonstrate that the concentration of the sugar (eg sucrose) needs to be varied with different substrates and/or fluorophores, depending on one or more of the solubility of the fluorophore, the physical texture (in particular the hydrophobicity) of the surface on to which the formulation is being sprayed, the droplet size, maintenance of the fluorophore in dispersion or solution, and the brightness of the formulation.

### Example 10

### Calcium Chloride as a Substitute for a Sugar

It was found that sugar in the fluorophore solution caused an increase in growth of bacteria and fungus. Calcium chloride was tested to check if it has hygroscopic properties and could replace sugar, hence eliminating the microbial growth.

A pyranine solution at 0.1% (w/v) solution was prepared with a concentration of 5 mM calcium chloride. This was sprayed on flowers and allowed to dry overnight. A control solution containing only 0.1% (w/v) pyranine alone was prepared and sprayed on flowers and allowed to dry overnight. A negative control with no spray was also used to monitor the experiment to determine vase life.

Flowers sprayed with 0.1% (w/v) pyranine solution with 5 mM calcium chloride did not exhibit any hygroscopic properties and also spread over the surface of the petal. The mist-like fluorescent spot formation was also not present on the petal of the flower as observed in the flower sprayed with the control solution, when observed under UV light.

### Example 11

### Fixatives

Different sealants were tested to preserve the fluorophore on flowers and to prevent the fluorophore from being rubbed away. Ironing Aid Starch (Coles Smart Buy), White Knight Crystal Clear Acrylic (White Knight) and Fixative Workable Mat (Micador) were tested for this purpose.

Flowers were pre-sprayed with Ironing Aid Starch, White knight Crystal Clear Acrylic and Fixative Workable Mat (on different flowers) and allowed to dry for an hour. All flowers were then sprayed with 0.1% (w/v) pyranine containing 5% (w/v) sugar and allowed to dry overnight. A negative control with no spray was also used to monitor the experiment to determine vase life.

Flowers pre-sprayed with Fixative Workable Mat and 0.1% (w/v) pyranine solution containing 5% (w/v) sugar emitted fluorescent blue spots, when excited by UV light. In addition, this did not prevent fluorophore rubbing, hence the Fixative Workable Mat was not considered ideal for this purpose.

Flowers pre-sprayed with honing Aid Atarch and 0.1% (w/v) pyranine solution containing 5% (w/v) sugar showed an increase in the size of the droplets formed and did not give mist-like spot formation. In addition, the spray did not preserve the fluorophore on the petal hence it was not considered further.

Flowers pre-sprayed with White Knight Crystal Clear Acrylic solution and 0.1% (w/v) pyranine solution containing 5% (w/v) sugar caused the wilting of the flower and affected the vase-life of the flower. Furthermore it did not preserve the fluorophore on the flower and the fluorophore rubbed off. Hence this was not considered further

Different sealants were also tested to preserve the fluorophore on the flower and to prevent the fluorophore from being rubbed away. Ironing Aid Starch, White Knight Crystal Clear Acrylic and Fixative Workable Mat were tested for this purpose

Flowers were pre-sprayed with 0.1% (w/v) pyranine containing 5% (w/v) sugar and allowed to dry for an hour. These flowers were then post sprayed with Ironing Aid Starch, White Knight Crystal Clear Acrylic and Fixative Workable Mat, where each spray went on different flowers sprayed with the fluorophore. A negative control with no spray was also used to monitor the experiment to determine vase life.

Flowers post sprayed with Ironing Aid Starch did not upset the mist-like fluorescent green droplet formation when excited by UV light; however it did not preserve the fluorophore on the flower. The fluorophore rubbed off on the hand, hence this spray was not considered.

Flowers post sprayed with White Knight Crystal Clear Acrylic showed wilting of the flower and reduced vase life of the flower. The fluorophore again was not preserved using this spray and rubbed off on the hand and was hence not further used.

Flowers post-sprayed with Fixative Workable Mat did not upset the mist-like fluorescent green droplet formation; however it did not prevent the fluorophore from being rubbed off from the flower. Hence this spray was not further used.

Flowers were also sprayed with 0.1% (w/v) pyranine solution and immediately sprayed with Ironing Aid Starch solution. This was allowed to dry overnight and observed the next day. A negative control with no spray was also used to monitor the experiment to determine vase life.

The fluorophore was not preserved on the flower even after it was sprayed with Ironing Aid Starch solution. The fluorophore rubbed off on the hand; hence this was not used further.

### Example 12

### Effect of Sugar Concentration

The amount of sugar in the fluorophore solution promoted the formation of bacteria and fungus and also caused the fluorophore solution to be sticky. Hence the effect of lower concentrations of sugar was tested, in order to determine the concentrations at which mist like droplet formation of the fluorophore solution was retained.

0.1% (w/v) pyranine solution was prepared with 5% (w/v), 1% (w/v), 0.5% (w/v), and 0.1% (w/v) sugar. 0.1% (w/v) pyranine solution with no sugar was also prepared and used as control solution. The different solutions were sprayed on flowers and were allowed to dry overnight. A negative control with no spray was also used to monitor vase life.

It was observed that 0.5% (w/v) sugar gave a similar mist-like fluorescent green droplet formation as compared to the fluorophores solution with 1% (w/v) and 5% (w/v) sugar, when excited by UV light. The control solution with no sugar and the 0.1% (w/v) pyranine solution with 0.1% (w/v) sugar did not form mist-like fluorescent green droplets and the fluorophore spread through-out the surface of the flower, when observed under UV light. The amount of stickiness in the fluorophore solution with 0.5% (w/v) sugar was considerably reduced as compared to 5% (w/v) sugar. Hence 0.5% (w/v) sugar was used in all the future experiments.

### Example 13

### Use of Polymers and Sealants Present in Hair Spray

Hair spray (Taft) was tested to check if it could preserve the fluorophore on the flower and prevent it from being rubbed off.

Flowers were sprayed with 0.1% (w/v) pyranine solution with 0.5% (w/v) sucrose, and allowed to dry for three hours. The flowers were then sprayed with hair spray and allowed to dry for four hours. A negative control with no spray was also used to monitor the experiment in order to determine vase life.

The hair spray was found to cause the fluorophore to emit fluorescent blue colour, when excited by UV light. It furthermore did not prevent the rubbing of the fluorophore from the hand. Hence the hair spray was not used in future experiments

### Example 14

### Use of PVA as a Sealant

Poly-vinyl alcohol (PVA) was also investigated to determine if it prevented the rubbing of the fluorophore from the flower.

Fluorophore solution was prepared with 0.1% (w/v) pyranine, 0.5% (w/v) sucrose and with varying concentrations of PVA: 1% (w/v), 0.5% (w/v) and 0.1% (w/v). Fluorophore solution with 0.1% (w/v) pyranine and 0.5% (w/v) sucrose was used as a control solution. The various solutions were prepared and sprayed on flowers and allowed to dry overnight. Lilies, carnations and chrysanthemums were used in this experiment. A negative control with no spray was also used to monitor the experiment to determine vase life.

The fluorophore solution with 1% (w/v), 0.5% (w/v) and 0.1% (w/v) of PVA did not prevent the rubbing off of the fluorophore on all three different varieties of flowers. The amount of fluorophore that rubbed off from flowers with sturdy petal (carnations) was less compared to those flowers with fragile petals (lilies).

### Example 15

### Use of Artist's Fixative as a Sealant

Artist Fixative (Winsor and Newton) was tested to check if the fluorophore could be preserved on the flower and could prevent the rubbing off of the fluorophore.

Flowers were sprayed with 0.1% (w/v) pyranine solution with 0.5% (w/v) sugar. This was immediately sprayed with Artist Fixative and allowed to dry for two hours. This experiment was performed on roses, carnations and chrysanthemums. A negative control with no spray was also used to monitor the experiment to determine vase life.

It was observed that the fluorophore did not rub off when it was rubbed gently as seen in all three varieties of flowers. The fluorophore, however rubbed off if the petals were rubbed harshly, as seen in all three variety of flowers.

### Example 16

### Use of Bicarbonate to Maintain pH

Pyranine was found to emit a fluorescent green solution at a higher pH when excited with UV light. Sodium bicarbonate was observed to increase the pH of the solution. Sodium bicarbonates was added to pyranine solution to maintain a higher pH so as to ensure that the fluorescence emitted from the flower remains green.

Fluorophore solution was prepared with 0.1% (w/v) pyranine, 0.5% (w/v) sugar and 0.1% (w/v) sodium bicarbonate. This was sprayed on flowers and allowed to dry overnight. A positive control with only 0.1% (w/v) pyranine, 0.5% (w/v) sugar and without sodium bicarbonate was also tested. A negative control with no spray was also used to monitor vase life.

The flowers sprayed with the fluorophore solution containing 0.1% (w/v) sodium bicarbonate gave green coloured fluorescence when excited with UV-light. This was similar to the positive control and it did not affect the vase-life of the flower. Hence it was used further in all other experiments.

### Example 17

### Fluorescence is Stable after Application of an Acidic Solution

It was observed from previous experiments that a shift in pH when excited with UV light caused the colour of the fluorescence emitted from the fluorophore sprayed on the flowers to change. Flowers were sprayed with a solution which had a pH of 7.72 and later sprayed with dilute HCl solution to observe any change.

Flowers sprayed with a solution containing 0.1% (w/v) pyranine, 0.5% (w/v) sugar and 0.1% (w/v) sodium bicarbonate which had a pH of 7.72. This was allowed to dry overnight. The flowers were then sprayed with 1 mM HCl, 1.5 mM HCl and 2 mM HCl solution. A set of control flowers was observed where they were only sprayed with the fluorophore and not sprayed with dilute HCl solution.

The fluorescence emitted by the flowers sprayed with the fluorophore on the first day was green when excited by UV light. However there was no change in colour to fluorescent blue when sprayed with dilute 1 mM HCl, 1.5 mM HCl and 2 mM HCl solution, as observed under UV light. The flowers emitted green fluorescence even after being sprayed with dilute HCl solution, when excited by UV light. Hence this experiment was not further considered.

### Example 18

### Use of Methyl Paraben as an Anti-Microbial Agent

The pyranine solution containing sugar enhanced the growth of bacteria and fungus; hence an anti-microbial agent, methyl paraben, was tested on the flowers.

The fluorophore solution was made up with 0.1% (w/v) pyranine, 0.5% (w/v) sugar and 0.1% (w/v) of methylparaben, in 10% (v/v) ethanol. This was sprayed on flowers and allowed to dry overnight. A negative control with no spray was also used to monitor vase life.

The fluorescent green colour emitted by the fluorophore was preserved when excited by UV light, however the solution caused wilting of the flowers and reduced the vase-life of the flower, hence this additive was not considered.

### Example 19

### Effect of Different Sugars

The effectiveness of different type of sugars on the droplet formation of the fluorophore solution was tested. Sucrose, glucose, fructose and mannitol were used for this purpose.

Fluorophore solution containing 0.1% (w/v) pyranine, 0.1% (w/v) sodium bicarbonate and 0.5% (w/v) of sucrose were prepared. Similar fluorophore solutions were prepared with the different sugar, namely glucose, fructose or mannitol. A negative control with no spray was also used to monitor the experiment to determine vase life.

Sucrose and fructose gave similar results with mist-like droplet formation all over the flower. Green fluorescence was emitted from the fluorophore in both cases, when excited by UV light. With glucose, the amount of fluorophore sticking onto the surface of the petal decreased. Similar green fluorescence was observed even in the case of glucose, when excited by UV light. With mannitol, there was an increase in the size of droplet formation, although the fluorophore emitted a fluorescent green colour, when excited by UV light. Sucrose was used for all future experiments.

### Example 20

### Sterilisation of Sugar Solutions

Fluorophore solution containing sugar caused the increase in growth of bacteria and fungus. Hence, the fluorophore solutions were autoclaved and the effect of autoclaving on droplet formation and vase-life of the cut flower were tested.

Fluorophore solution was prepared containing 0.1% (w/v) pyranine, 0.5% (w/v) sucrose, 0.1% (w/v) sodium bicarbonate. A similar solution was prepared with the same concentration of fructose. A small quantity of both the solutions prepared was kept aside for future use. Both the solutions were autoclaved at 121° C. in an Atherton Squirrel autoclave. This was then allowed to cool to room temperature and sprayed on flowers. Solutions which were kept aside previously (and which were not auto-claved) were also sprayed onto flowers. A negative control with no spray was also used to monitor vase life.

The fluorophore solution containing fructose appeared dark brown after it was autoclaved, whereas the fluorophore solution containing sucrose appeared to be fluorescent green. When the autoclaved fluorophore solution containing sucrose was sprayed on flowers, the droplets appeared to be bigger and the droplets had a slightly yellow appearance when excited by UV-light. A similar effect was observed when the autoclaved fluorophore solution containing fructose was sprayed on flowers. The vase life of the flower was not affected by the autoclaved solution.

### Example 21

### Preservatives

Bacterial and fungal growth was observed in the fluorophore solution containing sugar. Hence the preservatives, sodium benzoate and diazolidinyl urea were tested.

The fluorophore solution prepared contained 0.1% (w/v) pyranine, 0.5% (w/v) sucrose, 0.1% (w/v) sodium bicarbonate, and 0.1%(w/v) sodium benzoate. The solution to test the effect of diazolidinyl urea contained 0.1% (w/v) pyranine, 0.5% (w/v) sucrose, 0.1% (w/v) sodium bicarbonate, and 0.1%(w/v) diazolidinyl urea. Fluorophore solution containing only 0.1% (w/v) pyranine, 0.5% (w/v) sucrose, 0.1% (w/v) sodium bicarbonate was prepared and used as positive control. All the solutions were sprayed on lilly chrysanthemums and allowed to dry overnight. A negative control with no spray was also used to monitor the experiment to determine vase life.

A very strong smell was given by the fluorophore solution containing diazolidinyl urea, when it was being prepared. The flowers which were sprayed with the fluorophore solution which contained diazolidinyl urea, when excited by a UV light gave fluorescent green droplets. The droplet formation was similar to that of the positive control. The flowers which were sprayed with the fluorophore solution which contained sodium benzoate, gave fluorescent green droplets when excited by UV light. The vase life of the flowers was not affected when they were sprayed with fluorophore solution which contained diazolidinyl urea or sodium benzoate. Hence 0.1% (w/v) sodium benzoate was used in further experiments.

### Example 23

### Sugar Concentration

The effect of sugar concentration was further tested with the components sodium benzoate and sodium bicarbonate in the fluorophore solution.

The fluorophore solution prepared contained 0.1% (w/v) pyranine, 0.5% (w/v) sucrose, 0.1% (w/v) sodium bicarbonate and 0.1% (w/v) sodium benzoate. Fluorophore solution with a higher concentration of sugar 1% (w/v) sugar, 1.5% (w/v) sugar, 2% (w/v) sugar was also prepared and tested. The different solutions were sprayed on gerberas and allowed to dry overnight. A negative control with no spray was also used to monitor the experiment to determine vase life.

When the flowers sprayed with fluorophore solution with 0.5% (w/v) sucrose was compared with flowers sprayed with fluorophore solution containing 1% (w/v) sugar, 1.5% (w/v) sugar, 2% (w/v) sugar, it was observed that the flowers sprayed with 2% (w/v) sugar gave more droplets, as observed under the UV-lights. The amount of droplets sticking on to the petals of the flower increased with increasing concentration of the sugar in the fluorophore solution. However the flower sprayed with fluorophore solution containing 2% (w/v) sugar was extremely sticky when touched by hand due to the high sugar concentration. This was not observed in flowers sprayed with fluorophore containing 1.5% (w/v) sugar, 1% (w/v) sugar, and 0.5% (w/v) sugar. Hence fluorophore solution with 1.5% (w/v) sugar was used in further experiments. The vase life of the flowers were not affected by the high concentration of sugar.

### Example 24

### Uptake of Fluorophore

Freshly cut stems of flowers (roses and chrysanthemums) were prepared and the ends of the stems placed in a solution of 0.1% (w/v) pyranine, 0.5% (w/v) sucrose. The cut end of the flowers was left in the solution overnight and uptake of the fluorophore viewed under UVA light.

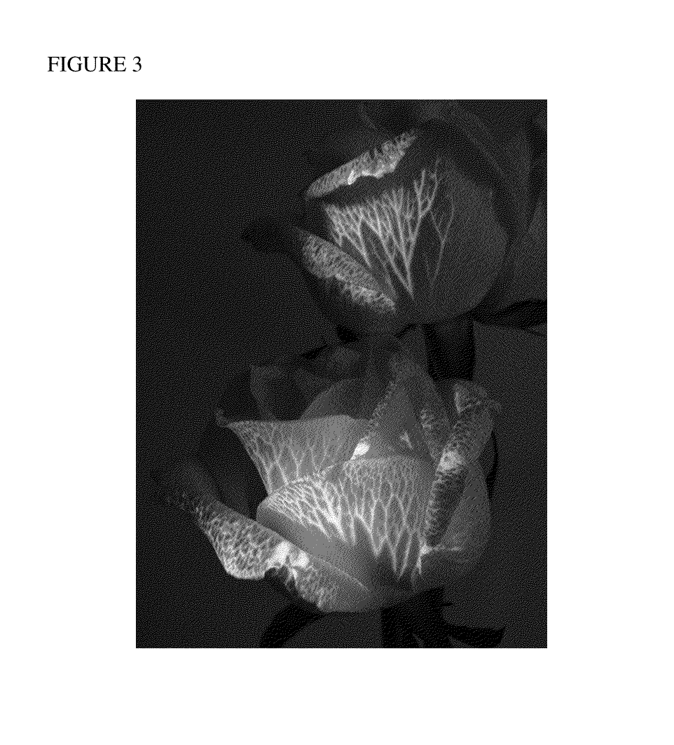

FIG. 3 shows uptake of the fluorophore in a rose flower. The veins in the petals of the flower demonstrate fluorescence. The fluorophore is moved to the flowers, through the stem, in the transpiration stream.

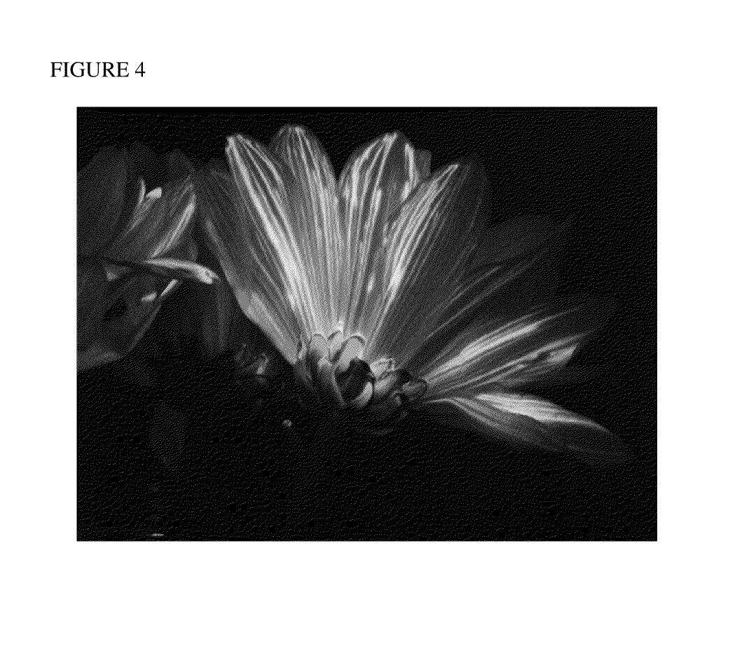

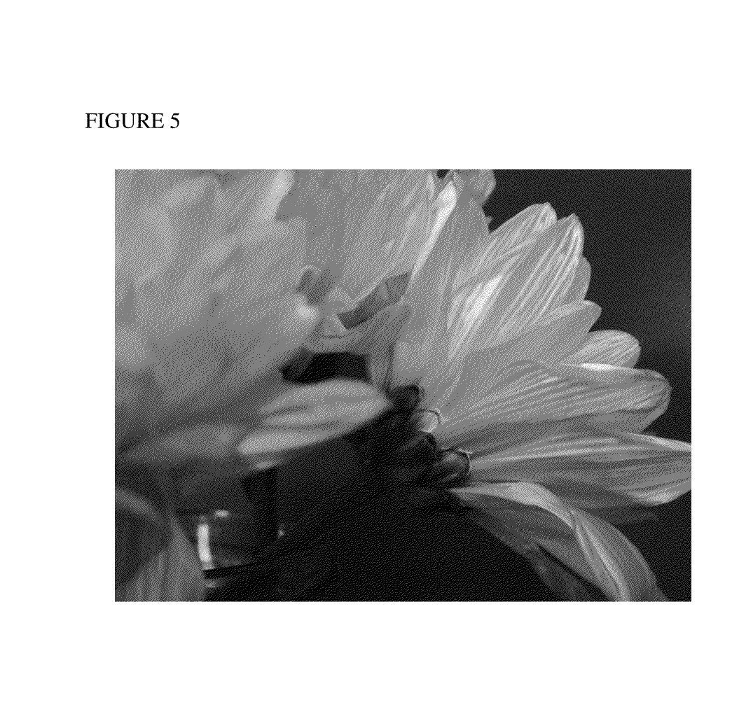

FIGS. 4 and 5 shows uptake of the fluorophore in a chrysanthemum flower. The veins in the petals of the flower demonstrate fluorescence. Once again, the fluorophore is moved to the flowers, through the stem, in the transpiration stream.

### Example 25

### Fluorescent Compositions

Examples of green, red and blue fluorescent formulations are as follows:

GREEN: 0.1% pyranine (8-hydroxypyrene-1,3,6-trisulfonic acid)
1.5% sugar
0.1% sodium bicarbonate
0.1% sodium benzoate
pH 7.5
Aqueous Formulation

RED: 0.07% Eu, 2-thenoyltrifluoroacetone triphenylphosphine oxide.
2.0% sugar
0.1% sodium bicarbonate
0.1% sodium benzoate
pH 7.5
The fluorescent agent is dissolved in ethylene glycol at 0.7%, to which is then added water to give the final concentration of 0.07%.

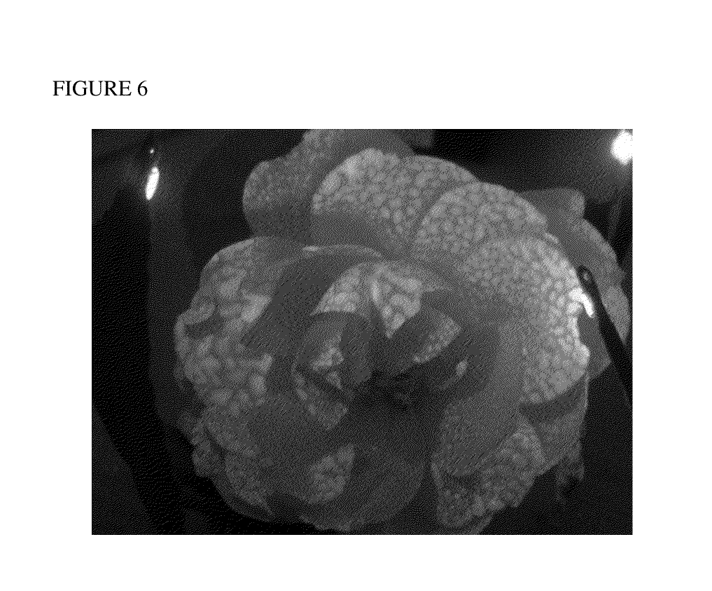

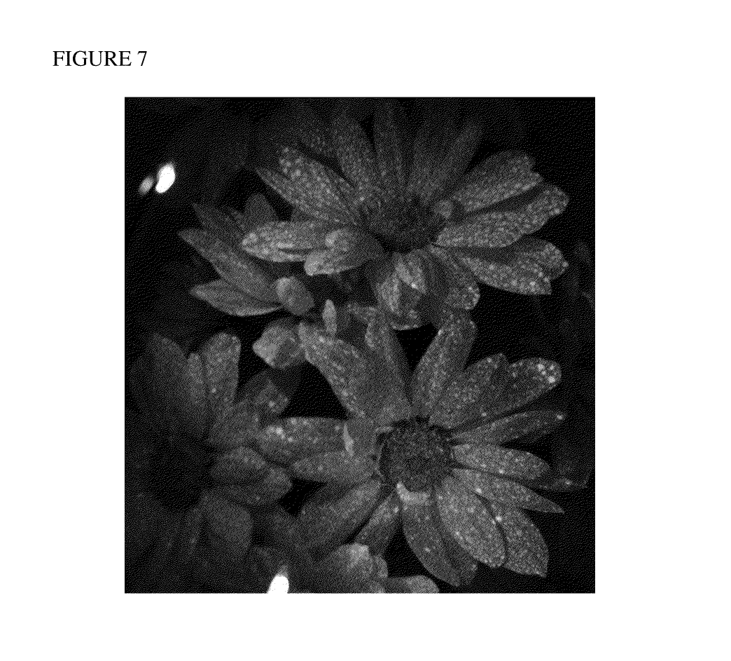

FIG. 6 shows use of a red formulation on a flower when excited by UV light. FIG. 7 shows another use of a red formulation on a flower when excited by UV light.

BLUE: 0.03% 4,4′-bis(2-benzoxazolyl)stilbene.
1.5% sugar
0.1% sodium bicarbonate
0.1% sodium benzoate
pH 7.5
The fluorescent agent is dissolved in ethylene glycol at 0.3%, to which is then added water to give the final concentration of 0.03%.

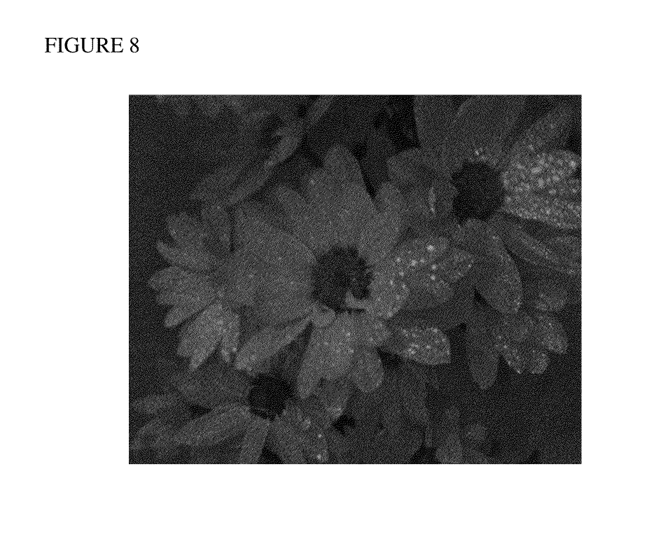

FIG. 8 shows use of a blue formulation on a flower when excited by UV light.

### Example 25

### Kits

A kit for spraying a fluorophore onto the surface of an object is as follows:

(i) Pump action atomizer container a spray solution as follows: 0.1% (w/v) pyranine, 0.5-1.5% (w/v) sucrose, 0.1% (w/v) sodium bicarbonate and 0.1% (w/v) sodium benzoate; and
(ii) Optionally instructions for spraying the solution onto an object.

A kit for spraying a fluorophore onto the surface of leaves and/or flowers in conjunction with use of an illumination device is as follows:

(i) Pump action atomizer container a spray solution as follows: 0.1% (w/v) pyranine, 0.5-1.5% (w/v) sucrose, 0.1% (w/v) sodium bicarbonate and 0.1% (w/v) sodium benzoate;
(ii) Optional instructions for spraying solution onto leaves and/or flowers; and
(iii) One or more of a vase, a corsage or a lapel flower, each of which has incorporated one or more UV emitting LEDs.

A kit for loading a fluorophore into a plant is as follows:

(i) An uptake solution as follows: 0.1% (w/v) pyranine and 0.5-1.5% (w/v) sucrose; and
(ii) Optional instructions for loading the fluorophore into the plant using the uptake solution.

A kit for loading a fluorophore into a plant is as follows:

(i) An uptake solution as follows: 0.1% (w/v) pyranine and 0.5-1.5% (w/v) sucrose;
(ii) Optional instructions for loading the fluorophore into the plant using the uptake solution;
(iii) One or more of a vase, a corsage or a lapel flower, each of which has incorporated one or more UV emitting LEDs.

Kits for use with a red and/or blue formulation as described herein may be prepared in a similar fashion.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1-35. (canceled)

36. A fluorescent marking composition for marking a plant and/or a part thereof, the composition comprising a polycyclic aromatic fluorophore.

37. The fluorescent marking composition according to claim 36, wherein the polycyclic aromatic fluorophore comprises one or more of a pyrene-based fluorophore, a stilbene-based fluorophore, and a metal ion complex.

38. The fluorescent marking composition according to claim 36, wherein the polycyclic aromatic fluorophore comprises a pyrene sulphonate.

39. The fluorescent marking composition according to claim 38, wherein the polycyclic aromatic fluorophore comprises 8-Hydroxy-1,3,6-pyrenetrisulfonic and/or a salt or a fluorescent derivative thereof.

40. The fluorescent marking composition according to claim 36, wherein the polycyclic aromatic fluorophore comprises a substituted benzoxazolyl stilbene.

41. The fluorescent marking composition according to claim 40, wherein the polycyclic aromatic fluorophore comprises a 4,4′-Bis(5-methyl-2-benzoxazolyl)stilbene and/or a salt or a fluorescent derivative thereof.

42. The fluorescent marking composition according to claim 36, wherein the polycyclic aromatic fluorophore comprises a triphenylphosphine oxide and/or a thenoyltrifluoroacetone complexed to a metal ion.

43. The fluorescent marking composition according to claim 42, wherein the polycyclic aromatic fluorophore comprises Eu, 2-thenoyltrifluoroacetone triphenylphosphine oxide and/or a salt or a fluorescent derivative thereof.

44. The fluorescent marking composition according to claim 36, wherein the polycyclic aromatic fluorophore comprises an emission wavelength in a range selected from one of 450 to 495 nm, 490 to 560 nm or 620 to 750 nm upon irradiation with light of an excitation wavelength.

45. The fluorescent marking composition according to claim 36, wherein the concentration of the polycyclic aromatic fluorophore is less than 0.5% (w/w).

46. The fluorescent marking composition according claim 36, wherein the composition comprises a saccharide.

47. The fluorescent marking composition according to claim 46, wherein the concentration of the saccharide is 5% or less (w/w).

48. The fluorescent marking composition according to claim 46, wherein the saccharide comprises a disaccharide and/or a monosaccharide.

49. The fluorescent marking composition according to claim 46, wherein the saccharide comprises sucrose.

50. The fluorescent marking composition according to claim 36, wherein the composition comprises a substantially aqueous composition.

51. An arrangement of one or more flowers and/or plants, the one or more flowers and/or plants comprising a fluorescent marking composition according to claim 36 applied to a surface of the one or more flowers and/or plants.

52. An arrangement of one or more flowers and/or plants, the one or more flowers and/or plants comprising a fluorescent marking composition according to claim 36, the fluorescent marking composition being taken up by the one or more flowers and/or plants.

53. A combination product, the combination product comprising the following components:
a fluorescent marking composition according to claim 36; and
a spraying and/or an atomizing device.

54. A method of marking a plant and/or part thereof with a fluorescent composition, the method comprising applying a fluorescent marking composition according to claim 36 to a surface of the plant and/or part thereof.

55. A method of producing a fluorescent plant and/or or a part thereof, the method comprising introducing a fluorescent marking composition according to claim 36 into the plant and/or a part thereof by uptake of the fluorescent marking composition by the plant and/or part thereof.
